(19) United States
(12) Patent Application Publication
(10) Pub. No.: US 2011/0159108 A1
(43) Pub. Date: Jun. 30, 2011

(54) GERM GUARD LID AND METHODS FOR USING GERM GUARD LID

(76) Inventors: Michael J. Maczuzak, Bratenahl, OH (US); Kathryn Jeanne Neiheiser, Strongsville, OH (US); Cherry Bochmann, Bay Village, OH (US)

(21) Appl. No.: 12/882,314

(22) Filed: Sep. 15, 2010

Related U.S. Application Data
(60) Provisional application No. 61/290,089, filed on Dec. 24, 2009.
(60) Provisional application No. 61/321,476, filed on Apr. 6, 2010.

(51) Int. Cl. A61K 33/38; B65D 90/00; A61K 33/30; A61P 31/10; A61P 31/04; A61P 31/00; A61P 31/12

(52) U.S. Cl. 424/618; 206/524.6; 424/641

(57) ABSTRACT

Described herein is a beverage lid that is itself treated with and/or impregnated with antimicrobial material to prevent the spread of contaminants and methods of making and using the same. The beverage lid can prevent spread of contaminants, including communicable diseases like influenza, the common cold, streptococcus or staphylococcus. For example, when a person carrying germs, like influenza, touches a beverage lid, the antimicrobial properties of the beverage lid can combat any contamination of the beverage lid by the germs. Another person can drink from the beverage lid that is substantially germ free.

### CROSS-REFERENCE TO RELATED CASES

The present application claims the priority of and expressly incorporates by reference the following applications: U.S. Provisional Patent Application Ser. No. 61/290,089, “GERM GUARD LABEL AND METHODS FOR USING A GERM GUARD LABEL,” which was filed on Dec. 24, 2009; U.S. Provisional Patent Application Ser. No. 61/321,476, “GERM GUARD LABEL AND METHODS FOR USING A GERM GUARD LABEL,” which was filed on Apr. 6, 2010.

### TECHNICAL FIELD

Described herein are beverage lids with antimicrobial features and, in particular, systems and methods employing beverage lids with antimicrobial features to prevent disease transmission.

### BACKGROUND

Consumers purchase millions of beverage containers each day, including take out cups, cans and bottles. Beverage containers can be purchased, for example, from takeout counters, from vending machines or from grocery stores. Even before reaching the store, beverage containers can be contaminated with dirt and grime from the packaging and shipping process. Once they reach the store, beverage containers can be contaminated with disease-causing germs from employees who touch the beverage containers.

For example, an employee may come to work at a store that sells beverages while suffering from a cold, influenza, or other contagious disease. When that employee touches the beverage container (e.g., places a lid on a cup of coffee or grabs a beer bottle from a refrigerator), the beverage container can become contaminated with germs. When the purchaser drinks the beverage, the germs are transferred from the beverage container to the purchaser.

### SUMMARY

The following presents a simplified summary of the invention in order to provide a basic understanding of some aspects of the invention. This summary is not an extensive overview of the invention and is not intended to identify key/critical elements of the invention or to delineate the scope of the invention. Rather, the sole purpose of this summary is to prevent some concepts of the invention in a simplified form as a prelude to the more detailed description that is presented later.

Described herein is a beverage lid that is itself treated with and/or impregnated with antimicrobial material to prevent the spread of contaminants and methods of making and using the same. The beverage lid can prevent spread of contaminants, including communicable diseases like influenza, the common cold, streptococcus or staphylococcus. For example, when a person carrying germs, like influenza, touches a beverage lid, the antimicrobial properties of the beverage lid can combat any contamination of the beverage lid by the germs. Another person can drink from the beverage lid that is substantially germ free.

To the accomplishment of the foregoing and related ends, the invention comprises the features hereinafter fully described and particularly pointed out in the claims. The following description sets forth in detail certain illustrative aspects and implementations of the invention. These are indicative, however, of but a few of the various ways in which the principles of the invention may be employed. Other objects, advantages and features of the invention will become apparent from the following detailed description of the invention.

### BRIEF DESCRIPTION OF THE DRAWINGS

FIG. 1 is an exemplary illustration of a beverage lid according to an embodiment.

### DETAILED DESCRIPTION

Described herein are beverage lids with antimicrobial features and methods for their preparation and methods for their use to prevent transmission of germs and other contaminants from a beverage lid to a human.

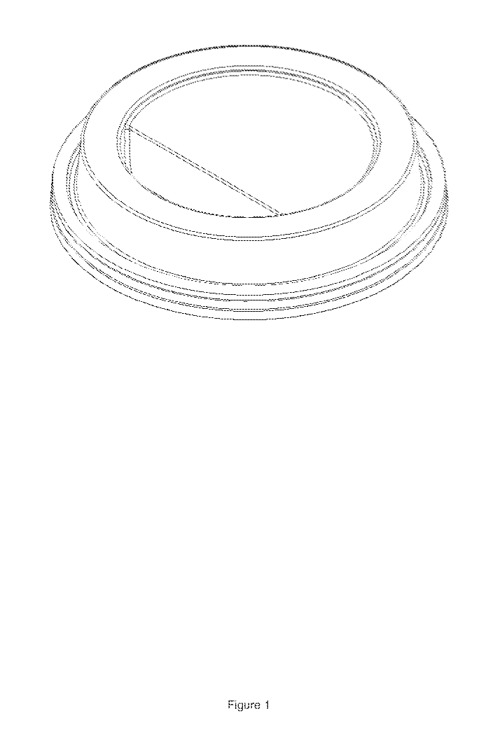

The term “beverage lid” as used herein can include a lid for any beverage container (e.g., a lid for a take-out cup, including a hot drink cup or a cold drink cup). An exemplary illustration of a beverage lid for a hot drink cup can be found in FIG. 1. However, the embodiment in FIG. 1 is not meant to be limiting. A beverage lid as described herein an also include, for example, a cup (e.g., a hot cup or a cold cup), a lid for a cup, a cup and a lid together, an aluminum can, a bottle, etc. A lid for a take-out coffee cup will be described herein for exemplary purposes, but it is not to be construed as limiting. The term “contaminant” as used herein can include, for example, germs, like bacteria or viruses, fungi, heavy metals and/or dirt.

A beverage lid can be made of a polymer material. The polymer material can be generally polystyrene (PS). Additionally or alternatively, the polymer can be generally polylactide. The material can include traces of other materials. For example, the polymer material can include at least one of polycarbonate (PC), polypropylene (PP), polyethylene (e.g., high-density polyethylene (HDPE), low density polyethylene (LDPE) or polyethylene terephthalate (PET)), polyvinyl chloride (PVC or vinyl), polystyrene (PS), etc. The polymer material can, additionally or alternatively, include a biopolymer (e.g., cellulose or cellulose acetate). The material can include additives that can enhance various properties of the beverage lid, for example, color. Materials used in the polymer material can be any material approved for food contact by the U.S. Food and Drug Administration (FDA).

The beverage lid can prevent transmission of contaminants. According to an embodiment, the polymer material of the beverage lid can have an antimicrobial agent directly incorporated. In other words, the polymer material can be impregnated with the antimicrobial agent. According to another embodiment, an antimicrobial agent can be adsorbed onto the surface of the polymer material of the beverage lid. In other words, the beverage lid can be treated with the antimicrobial agent. The antimicrobial agent can be impregnated within and/or adsorbed on the surface of any the polymer material that can be formed into any beverage lid as described herein; for example, a lid, as illustrated in FIG. 1, a cold cup lid or even a cold cup itself. For example, polymers used to form a cold cup (e.g. a disposable plastic cup) can be impregnated with the antimicrobial agent and/or can have the antimicrobial agent adsorbed onto the surface.

The antimicrobial agent can be one or more FDA-approved antimicrobial agents. The antimicrobial agents can include chemical antimicrobials, antioxidants, biotechnology products, antimicrobial polymers, natural antimicrobials or gas. The antimicrobial agents can prevent or limit the spread of contaminants.

The antimicrobial agent can include a biocidal (life-destroying) agent(s) and/or a biostatic agent(s). The biocidal agent(s) and/or biostatic agent(s) can slow and/or inhibit the growth or reproduction of microorganisms, including bacteria, molds, fungi, viruses and the like. The biocidal agent(s) can include an antimicrobial agent, such as a bacteriacide and/or a fungicide. The antimicrobial agent can inhibit growth of microorganisms and/or destroy microorganisms. The antimicrobial agent can also include an antiviral agent.

Any antimicrobial agent that is compatible with the polymer material of the beverage lid (e.g., PS) before fabrication and will retain its activity after fabrication can be used in connection with the beverage lid. For example, an antimicrobial agent used with the beverage lid can include a broad spectrum biocide and a wide ranging antimicrobial. The wide ranging antimicrobial can control and/or kill germs, including gram positive bacteria, gram negative bacteria, aerobic bacteria and anaerobic bacteria, viruses, fungi, etc.

For example, the antimicrobial agent can include triclosan, which is a common name for 2, 4, 4′-trichloro-2′-hydroxydiphenol or 50chloro-2-(2, 4,-dichlorophenoxy)phenol. Further for example, the antimicrobial agent can include silver or silver ion. The antimicrobial agent can, additionally or alternatively, include zinc or zinc ion. The antimicrobial agent can, additionally or alternatively, include iodine or iodine ion. The antimicrobial can, additionally or alternatively, include an amine group.

Antimicrobial agent(s) can prevent the spread of contaminants, for example, from human-to-human. For example, when a person infected with germs like influenza touches a beverage lid to place it on a cup, influenza germs can travel from the infected person's hand to the beverage container and beverage lid. A beverage lid with an antimicrobial agent with antiviral properties can free the beverage lid of contamination with influenza germs. Another person can drink from the uncontaminated beverage lid.

According to an embodiment, the beverage lid can be constructed in by providing a food-grade polymer material. For example, the material can be generally PS with additives. The material can be treated with an antimicrobial. According to an embodiment, the material can be impregnated with the antimicrobial agent. Additionally or alternatively, the material can be coated with the antimicrobial agent. For example, the antimicrobial agent can be adsorbed onto the surface of the material.

The antimicrobial agent can be any FDA-approved antimicrobial agent. The antimicrobial agent can be active against one or more of bacteria, viruses, fungi or any other contaminant.

The antimicrobial agent can include a biocidal and/or a biostatic agent(s). The biocidal and/or biostatic agent may be capable of slowing or inhibiting the growth or reproduction of microorganisms, for example triclosan, including bacteria, molds, fungi, viruses and the like. Additionally, the biocidal agent may be a biocide (life destroying), which may be a bacteriacide and fungicide (inhibiting the growth of microorganisms and destroying the microorganisms). The biocide may also be antiviral. The biocide may be a broad spectrum biocide, and generally, a wide ranging antimicrobial, inhibiting a broad range of microorganisms. The antimicrobial may be effective at controlling or killing bacteria, such as gram positive, gram negative, aerobic and anaerobic types, viruses, fungi, etc. Any biocide that is compatible with the material before fabrication and will retain its activity after fabrication may be used in connection with the beverage lid.

For example, the antimicrobial can be triclosan, which is a common name for 2, 4, 4′-trichloro-2′-hydroxydiphenol or 50chloro-2-(2, 4,-dichlorophenoxy)phenol. Further for example, the antimicrobial can contain silver or silver ion. The antimicrobial can, additionally or alternatively, contain zinc or zinc ion, iodine or iodine ion, and/or an amino group.

The material can be treated with the antimicrobial after the beverage lid is manufactured. Before the material is treated, the beverage lid can be cleaned to remove any contaminants from the production process. The beverage lid can be cleaned, for example, by a water rinse and/or a commercial cleanser. Additionally or alternatively, the beverage lid can be cleaned utilizing liquid acidic or alkaline cleaners. The beverage lid can be dried, for example, in an oven and/or spray-dried.

Additionally or alternatively, the beverage lid can be sanitized and/or sterilized to remove any germs from the surface of the beverage lid before being treated with the antimicrobial. Any method of sanitization can be used, including thermal sanitization, ultraviolet sanitization, x-ray sanitization, or chemical sanitization. For example, the object may be sanitized using dry heat, wherein the object is exposed to a high temperature (e.g., greater than 100 degrees Celsius) for a period of time (e.g., greater than 1 hour).

The beverage lid with antimicrobial properties can prevent transmission of germs, for example, from person to person. For example, when a person carrying germs (e.g., influenza) touches a beverage lid and puts it on a beverage container, he transfers germs from his hand to the beverage lid. The beverage lid with the antimicrobial (e.g., antiviral) properties neutralizes the germs and prevents the spread of germs to another person who drinks through the lid.

Other than in the operating examples, or where otherwise indicated, all numbers, values and/or expressions referring to quantities of ingredients, reaction conditions, etc., used in the specification and claims are to be understood as modified in all instances by the term “about.”

What has been described above includes examples of the present specification. It is, of course, not possible to describe every conceivable combination of components or methodologies for purposes of describing the present specification, but one of ordinary skill in the art may recognize that may further combinations and permutations of the present specification are possible. Accordingly, the present specification is intended to embrace all such alterations, modifications and verifications that fall within the spirit and scope of the appended claims. In addition, while a particular feature of the invention may have been disclosed with respect to only one of several embodiments, such feature may be combined with one or more other features of the other embodiments as may be desired and advantageous for any given or particular application. Furthermore, to the extent that the term “includes” is used either in the detailed description or the claims, such term is intended to be inclusive in a manner similar to the term “comprising” as “comprising” is interpreted when employed as a transitional word in a claim.

What is claimed is:

1. A beverage lid that prevents contaminant transmission, comprising:
a polymer material comprising polystyrene; and
an antimicrobial agent.

2. The beverage lid of claim 1, wherein the antimicrobial agent is impregnated into the polymer material.

3. The beverage lid of claim 1, wherein the antimicrobial agent is adsorbed to the surface of the polymer material.

4. The beverage lid of claim 1, wherein the antimicrobial agent comprises at least one of: silver, silver ion.

5. The beverage lid of claim 1, wherein the antimicrobial agent comprises at least one of: zinc or zinc ion.

6. The beverage lid of claim 1, wherein the antimicrobial agent comprises at least one of iodine or iodine ion.

7. The beverage lid of claim 1, wherein the antimicrobial agent comprises an amino group.

8. The beverage lid of claim 1, wherein the antimicrobial agent comprises at least one of an antibacterial agent, an antifungal agent or an antiviral agent.

9. The beverage lid of claim 1, wherein the antimicrobial agent comprises a biostatic agent.

10. An article, comprising:
a polymer material comprising polystyrene; and
an antimicrobial agent.

11. The article of claim 10, wherein the antimicrobial agent is impregnated within the polymer material.

12. The article of claim 10, wherein the antimicrobial agent is adsorbed to the surface of the polymer material.

13. A method for making a beverage lid, comprising:
providing a polymer material comprising polystyrene;
treating the polymer material with an antimicrobial agent;
shaping the polymer material into a beverage lid.

14. The method of claim 13, wherein treating the polymer material with the antimicrobial agent further comprises impregnating the polymer material with the antimicrobial agent.

15. The method of claim 13, wherein treating the polymer material with the antimicrobial agent further comprises adsorbing the antimicrobial agent to the polymer material.

16. The method of claim 13, further comprising cleaning the beverage lid.

17. The method of claim 13, further comprising sanitizing the beverage lid.

18. The method of claim 13, wherein the antimicrobial agent comprises at least one of an antibacterial agent, an antifungal agent or an antiviral agent.

19. The method of claim 13, wherein the antimicrobial agent comprises at least one of: silver, silver ion, zinc or zinc ion.

20. The method of claim 13, wherein the antimicrobial agent comprises at least one of a biocide or a biostatic.
